(19) United States
(12) Patent Application Publication
(10) Pub. No.: US 2026/0174636 A1
(43) Pub. Date: Jun. 25, 2026

(54) NATURAL DYE COLORING WITH IMPROVED COLOR INTENSITY

(71) Applicant: HENKEL AG & CO. KGAA, Düsseldorf (DE)

(72) Inventors: Nele DALLMANN, Hamburg (DE); Fabian STRASKE, Hamburg (DE); Skrollan KLAAS, Hamburg (DE); Jana Marie ROESEKE, Tornesch (DE); Thomas HIPPE, Appen (DE)

(21) Appl. No.: 19/123,755

(22) PCT Filed: Sep. 29, 2023
(86) PCT No.: PCT/EP2023/077008

§ 371 Date: Apr. 23, 2025

(30) Foreign Application Priority Data
Oct. 28, 2022 (DE) 10 2022 211 482.5

(51) Int. Cl. A61Q 5/06; A61Q 5/10

(52) U.S. Cl. CPC A61K 8/00; A61Q 5/065; A61Q 5/10

(57) ABSTRACT

A method for coloring keratin-containing fibers, in particular such as human hair, using a natural dye based upon anthocyanins and at least one glycosylase is described. The natural dye is also described.

The invention relates to a method for coloring keratin-containing fibers, in particular human hair, using a natural dye based upon anthocyanins and at least one glycosylase (E.C. 3.2), and to compositions for carrying out this coloring method.

The desire to change one's hair color is a great need for many consumers. To satisfy this need, the cosmetic industry provides a diverse range of products. Hair coloring agents that achieve particularly long-lasting coloring with high coverage are usually oxidation dyes. These use oxidizing agents, which can damage the hair structure. Certain cationic direct azo dyes are also capable of producing hair color changes with excellent fastness properties. The azo dyes mentioned are synthetic dyes. However, a growing number of consumers are demanding hair coloring agents and hair coloring methods based upon natural dyes, even though these agents and methods are often inferior to the aforementioned agents and methods in terms of fastness properties, coverage, and color variety.

### PRIOR ART

The published patent application WO2019244920A1 discloses the production of color pigments obtained by pretreating anthocyanin-containing plant parts with cellulase at a pH of 4.5. The enzyme is then washed out, and enzyme-free pigments are obtained.

### OBJECT

The object of the present invention was to provide a method for coloring keratin-containing fibers, in particular human hair, using naturally occurring substances. A further object of the present invention was to provide a method for coloring keratin-containing fibers, in particular human hair, using naturally occurring substances with which colorings with good coloring properties are achieved, in particular with high color intensity, good wash-, light-, and/or rubbing fastness, good homogeneity, and a good balancing capacity, i.e., a low selectivity between damaged and undamaged regions along the keratin fibers.

Surprisingly, it was found that, by combining at least one anthocyanin-containing plant part and at least one glycosylase (E.C. 3.2) located outside the plant part, coloring compositions for keratin fibers can be obtained that achieve colorings with good application properties.

A first subject matter of the present invention is a method for the non-oxidative coloring of keratinous fibers, in particular human hair, in which a composition containing at least one anthocyanin-containing plant part and at least one glycosylase (E.C. 3.2) located outside the plant part is applied to the keratin fibers and rinsed off again after an exposure time.

Coloring methods and coloring compositions according to the invention are characterized in that the at least one glycosylase is located outside the anthocyanin-containing plant part. This means that the at least one glycosylase is provided separately from the composition containing the at least one anthocyanin-containing plant part. The glycosylases provided separately according to the invention therefore do not include those glycosylases that are naturally present in the anthocyanin-containing plant parts used.

### Glycosylases (E.C. 3.2)

Without wishing to be bound to this theory, it is assumed that the addition of at least one glycosylase (E.C. 3.2) to a composition containing at least one anthocyanin-containing plant part causes the aglycone of the anthocyanin, i.e., the anthocyanidin, to be produced in situ, which immediately effects the coloring of the keratin fiber.

In a preferred embodiment of the invention, the at least one glycosylase (E.C. 3.2) is selected from at least one glycosidase (E.C. 3.2.1). Glycosidases (E.C. 3.2.1) are understood to mean enzymes that hydrolyze O-glycosyl components and S-glycosyl components.

Glycosidases (E.C. 3.2.1) preferred according to the invention are selected from a group of enzymes generally referred to as cellulases.

The term “cellulase” as used herein refers to enzymes that catalyze the hydrolysis of 1,4-beta-D-glucoside bonds that are present in cellulose (cellobiose) and/or lichenin and/or beta-D-glucans. Cellulases are often also able to hydrolyze the 1,4 bonds in beta-D-glucans, which also have 1,3 bonds in addition to the 1,4 bonds. Cellulases are able to break down cellulose to beta-glucose. Consequently, cellulases act in particular upon cellulose-containing or cellulose derivative-containing functional groups and catalyze their hydrolysis. The determining factor as to whether an enzyme is a cellulase in the context of the invention is its ability to hydrolyze 1,4-beta-D-glucoside bonds in cellulose.

The term “cellulase activity” is defined here as an enzyme that catalyzes the hydrolysis of 1,4-beta-D-glucoside bonds in beta-1,4-glucan (cellulose). Cellulose activity is measured using a standard method, e.g., as follows: Cellulases release glucose from CMC (carboxymethylcellulose). The samples are incubated under defined reaction conditions (100 mM sodium phosphate buffer pH 7.5, 40° C., 15 min) with a substrate (1.25 wt. % CMC). The reaction with p-hydroxybenzoic acid hydrazide (PAHBAH) in the presence of bismuth produces a yellow dye that can be determined photometrically at 410 nm. The prerequisite is an alkaline pH during the color reaction. The amount of sugar released corresponding to the coloration is a measure of enzyme activity (Lever, Anal. Biochem., 1972, 47 & 1977, 81).

Cellulases can be divided into three categories:
1. Endoglucanase (E.C. 3.2.1.4), also called endo-1,4-beta-glucanase, beta-1,4-glucanase, avicelase, beta-1,4-endoglucanhydrolase, endo-1,4-beta-D-glucanohydrolase, carboxymethylcellulase, or celludextrinase;
2. Cellulose-1,4-beta-cellobiosidase (non-reducing end) (E.C. 3.2.1.91), also called exoglucanase, 1,4-beta-cellobiohydrolase, 4-beta-D-glucan cellobiohydrolase (non-reducing end), avicelase, exo-1,4-beta-D-glucanase, or exocellobiohydrolase, releases cellobiose from the non-reducing ends of the B-D-glucan chains by hydrolysis of the (1->4)-beta-D-glucosidic bonds in cellulose and cellotetraose;
3. beta-glucosidase (E.C. 3.2.1.21), also called cellobiase, beta-D-glucoside glucohydrolase, amygdalase, or gentobiase, hydrolyzes terminal, non-reducing beta-D-glucosyl groups to release beta-D-glucose.

Suitable cellulases include those of bacterial or fungal origin. Chemically modified or protein-engineered mutants are included. Suitable cellulases are cellulases from the genera Bacillus, Pseudomonas, Humicola, Fusarium, Thielvia, Acremonium, e.g., the fungal cellulases from Humicola insolens, Myceliophthora thermophila, and Fusarium oxysporum, which are disclosed in U.S. Pat. Nos. 4,435,307, 5,648,263, 5,691,178, 5,776,757, and WO 89/09259. Particularly suitable cellulases can be alkaline or neutral cellulases with color care properties. Examples of such cellulases are cellulases that are described in EP 495257, EP 531372, WO 96/11262, WO 96/29397, and WO 98/08940. Other examples are cellulase variants as described in WO 94/07998, EP531315, EP 3212777, EP 3502243, EP 3653705, EP 3653706, U.S. Pat. Nos. 5,457,046, 5,686,593, 5,763,254, WO 95/24471, WO 98/12307 and WO 99/01544, and WO 2019/122520.

Examples of glycosylases and glycosidases with endo-1,4-glucanase activity (E.C. 3.2.1.4) are described in WO 2002/099091—for example, those having a sequence of at least 97% identity to the amino acid sequence of positions 1 to 773 of SEQ ID NO:2 of WO 2002/099091. A further example can comprise a GH44-xyloglucanase—for example, a xyloglucanase enzyme having a sequence of at least 60% identity to positions 40 to 559 of SEQ ID NO:2 of WO 2001/062903.

Other examples of cellulases suitable according to the invention include the GH45 cellulases described in WO 96/29397 and, in particular, variants thereof having substitution, insertion, and/or deletion at one or more of the positions corresponding to the following positions in SEQ ID NO:8 of WO 2002/099091:2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected from P 19A, G20K, Q44K, N48E, Q119H, or Q146R.

Commercially available cellulases include Celluzyme™, Carezyme™, Carezyme Premium™, Celluclean™ (e.g., Celluclean™ 5000L and Celluclean™ 4000T), Celluclean Classic™, Cellusoft™, Endolase®, Renozyme®, and Whitezyme™ (Novozymes A/S), Clazinase™ and Puradax HA™ (Genencor International Inc.), KAC-500 (B)™ (Kao Corporation), Revitalenz™ 1000, Revitalenz™ 2000, and Revitalenz™ 3000 (DuPont), as well as Ecostone® and Biotouch® (AB Enzymes).

In a further preferred embodiment of the invention, the at least one glycosidase (E.C. 3.2.1) is selected from endo-1,4-beta-glucanase (E.C. 3.2.1.4), beta-glucosidase (E.C. 3.2.1.21), exo-1,4-beta-glucosidase (E.C. 3.2.1.74), cellulose-1,4-beta-cellobiosidase (E.C. 3.2.1.176), exo-1,4-beta-D-glucanase (E.C. 3.2.1.91), and oligoxyloglucan reducing-end-specific cellobiohydrolase (E.C. 3.2.1.150), and mixtures of these enzymes.

In a further preferred embodiment of the invention, the at least one glycosidase (E.C. 3.2.1) is selected from endo-1,4-beta-glucanase (E.C. 3.2.1.4), beta-glucosidase (E.C. 3.2.1.21), and exo-1,4-beta-D-glucanase (E.C. 3.2.1.91), and mixtures of these enzymes.

A particularly preferred embodiment of the invention is characterized in that the coloring composition contains an enzyme mixture comprising at least one endo-1,4-beta-glucanase (E.C. 3.2.1.4), at least one beta-glucosidase (E.C. 3.2.1.21), and at least one enzyme selected from at least one exo-1,4-beta-D-glucanase (E.C. 3.2.1.91) and at least one oligoxyloglucan reducing-end-specific cellobiohydrolase (E.C. 3.2.1.150), and mixtures thereof.

A particularly preferred embodiment of the invention is characterized in that the coloring composition contains an enzyme mixture comprising at least one endo-1,4-beta-glucanase (E.C. 3.2.1.4), at least one beta-glucosidase (E.C. 3.2.1.21), and at least one exo-1,4-beta-D-glucanase (E.C. 3.2.1.91).

A further particularly preferred embodiment of the invention is characterized in that the coloring composition contains an enzyme mixture comprising at least one endo-1,4-beta-glucanase (E.C. 3.2.1.4) in a total amount of 20-35 wt. %, at least one beta-glucosidase (E.C. 3.2.1.21) in a total amount of 15-25 wt. %, and—in a total amount of 25-35 wt. %—at least one enzyme selected from at least one exo-1,4-beta-D-glucanase (E.C. 3.2.1.91) and at least one oligoxyloglucan reducing-end-specific cellobiohydrolase (E.C. 3.2.1.150), and mixtures thereof, wherein the indicated amounts are relative to the total weight of the enzyme mixture.

A further particularly preferred embodiment of the invention is characterized in that the coloring composition contains an enzyme mixture comprising at least one endo-1,4-beta-glucanase (E.C. 3.2.1.4) in a total amount of 20-35 wt. %, at least one beta-glucosidase (E.C. 3.2.1.21) in a total amount of 15-25 wt. %, and at least one exo-1,4-beta-D-glucanase (E.C. 3.2.1.91) in a total amount of 25-35 wt. %, wherein the indicated amounts are relative to the total weight of the enzyme mixture.

A further particularly preferred embodiment of the invention is characterized in that the coloring composition contains an enzyme mixture comprising at least one endo-1,4-beta-glucanase (E.C. 3.2.1.4) in a total amount of 20-35 wt. %, at least one beta-glucosidase (E.C. 3.2.1.21) in a total amount of 15-25 wt. %, furthermore-in a total amount of 25-35 wt. %—at least one enzyme selected from at least one exo-1,4-beta-D-glucanase (E.C. 3.2.1.91) and at least one oligoxyloglucan reducing-end-specific cellobiohydrolase (E.C. 3.2.1.150), and mixtures thereof, as well as at least one other enzyme with cellulase activity in a total amount up to 100 wt. %, wherein the indicated amounts are relative to the total weight of the enzyme mixture.

A further particularly preferred embodiment of the invention is characterized in that the coloring composition contains an enzyme mixture comprising at least one endo-1,4-beta-glucanase (E.C. 3.2.1.4) in a total amount of 20-35 wt. %, at least one beta-glucosidase (E.C. 3.2.1.21) in a total amount of 15-25 wt. %, and at least one exo-1,4-beta-D-glucanase (E.C. 3.2.1.91) in a total amount of 25-35 wt. %, as well as at least one other enzyme with cellulase activity in a total amount up to 100 wt. %, wherein the indicated amounts are relative to the total weight of the enzyme mixture.

A further particularly preferred embodiment of the invention is characterized in that the coloring composition contains, based upon its weight, at least one glycosylase (E.C. 3.2) in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

### Origin of the Anthocyanins

### a) Plants

A preferred embodiment of the invention is characterized in that the anthocyanin-containing plant used according to the invention is selected from Ribes nigrum, Euterpe oleracea (cabbage palm, açai), Aronia (chokeberry), Malus domestica, Solanum melongena, Fragaria, Vaccinium, Hibiscus, Sambucus nigra, Citrus sinensis, Rubus sect. Rubus, Rubus idaeus, Prunus avium, Prunus cerasus, Ribes rubrum, red cabbage (Brassica oleracea convar. capitata var. rubra L.), and Vitis vinifera. Of course, the coloring compositions according to the invention and used according to the invention can also contain plant parts of two or more different plants. In a particularly preferred embodiment of the invention, the anthocyanin-containing plant used according to the invention is selected from Ribes nigrum.

### b) Plant Parts

A preferred embodiment of the invention is characterized in that the anthocyanin-containing plant part is selected from berries, fruits, aggregate nut fruits, aggregate drupes, drupes, pomes, peels, cones, leaves, stems, branches, bark, trunk wood, seeds, or roots of an anthocyanin-containing plant. In a particularly preferred embodiment of the invention, the plant parts used are used in dried and crushed form. In an extremely preferred embodiment of the invention, berries are used as the anthocyanin-containing plant part, particularly preferably in dried and crushed form. In a further extremely preferred embodiment of the invention, the berries of Ribes nigrum are used as the anthocyanin-containing plant part, particularly preferably in dried and crushed form.

In a further extremely preferred embodiment of the invention, the at least one anthocyanin-containing plant part is used in powder form, particularly preferably as a water-soluble powder. Water solubility is understood here to mean that at least 0.3 wt. %, preferably at least 0.5 wt. %, of the powder dissolves clearly in water at 25° C.

In a further preferred embodiment of the invention, the composition used for coloring is characterized in that it contains, based upon its weight, at least one anthocyanin-containing plant part in a total amount of 0.05-20 wt. %, preferably 0.1-10 wt. %, more preferably 0.5-5 wt. %, particularly preferably 0.7-2 wt. %, extremely preferably 1-1.5 wt. %.

In a further extremely preferred embodiment of the invention, an extract of the anthocyanin-containing plant part is used rather than the at least one anthocyanin-containing plant part per se.

Plant parts preferred according to the invention from which extracts preferred according to the invention can be obtained are the berries, fruits, aggregate nut fruits, aggregate drupes, drupes, pomes, peels, cones, leaves, stems, branches, bark, trunk wood, seeds, or roots of an anthocyanin-containing plant.

Suitable extractants are water, in particular hot water with a temperature of 45-100° C., furthermore C₁-C₄ alkanols and C₂-C₄ polyols, in particular ethanol, isopropanol, n-propanol, ethylene glycol, 1,2-propanediol, glycerin, and 1,3-butylene glycol, and mixtures of these extractants, in particular mixtures of water and at least one C₁-C₄ alkanol, mixtures of water and at least one C2-C4 polyol, particularly preferably water/ethanol mixtures.

The extract itself can preferably be used in concentrated form, obtained by partial distillation of the extractant after extraction, as a viscous liquid. Other extracts preferred according to the invention are used in powder form, obtained by drying, preferably spray drying, the solvent-containing extract.

In a further preferred embodiment of the invention, the composition used for coloring is characterized in that it contains, based upon its weight, at least one anthocyanin-containing plant part extract in a total amount of 0.005-20 wt. %, preferably 0.01-5 wt. %, more preferably 0.1-2 wt. %, particularly preferably 0.2-1 wt. %, extremely preferably 0.3-0.6 wt. %.

In a further preferred embodiment of the invention, the composition used for coloring is characterized in that it contains, based upon its weight, at least one anthocyanin in a total amount of 0.001-2 wt. %, preferably 0.01-1 wt. %, more preferably 0.02-0.5 wt. %, particularly preferably 0.03-0.2 wt. %, extremely preferably 0.05-0.1 wt. %.

### Incubation Time

In this context, incubation time is understood to mean the time between the addition of the glycosylase solution to the composition containing the anthocyanin-containing plant part and the contact of the coloring composition with the keratin fibers to be colored. Surprisingly, it was found that the shortest possible incubation time is most suitable for the color result.

### Exposure Time

A feature of the coloring method according to the invention is that a composition containing at least one anthocyanin-containing plant part and at least one glycosylase (E.C. 3.2) located outside the plant part is applied to the keratin fibers and rinsed off again after an exposure time. This exposure time is preferably 1 to 60 minutes, more preferably 5 to 45 minutes, particularly preferably 20 to 35 minutes, extremely preferably 25 to 30 minutes.

After the exposure time for the composition according to the invention or preferred according to the invention has elapsed, the keratin fibers are rinsed with water in order to wash out the composition according to the invention or preferred according to the invention.

A preferred subject matter of the present invention is a method for the non-oxidative coloring of keratinous fibers, in particular human hair, in which a composition containing at least one anthocyanin-containing plant part and a second composition containing at least one glycosylase (E.C. 3.2) are mixed with one another, and this mixture is applied to the keratin fibers within a time of zero seconds to 10 minutes, preferably 10 seconds to 5 minutes, particularly preferably 15 to 60 seconds, and is rinsed off again after an exposure time of 1 to 60 minutes, more preferably 5 to 45 minutes, particularly preferably 20 to 35 minutes, extremely preferably 25 to 30 minutes.

What has been said regarding preferred embodiments of the coloring method according to the invention as defined in claim 1 applies mutatis mutandis to preferred embodiments of this coloring method preferred according to the invention with the stated incubation and exposure times.

A preferred subject matter of the present invention is a method for the non-oxidative coloring of keratinous fibers, in particular human hair, in which a composition containing at least one anthocyanin-containing plant part and a second composition containing at least one glycosylase (E.C. 3.2) are mixed with one another, and this mixture is applied to the keratin fibers within a time of zero seconds to 10 minutes, preferably 10 seconds to 5 minutes, particularly preferably 15 to 60 seconds, and is rinsed off again after an exposure time of 1 to 60 minutes, more preferably 5 to 45 minutes, particularly preferably 20 to 35 minutes, extremely preferably 25 to 30 minutes, wherein the ready-to-use coloring composition has a temperature of 15-50° C., preferably 20-40° C., preferably 25-35° C.

What has been said regarding preferred embodiments of the coloring method according to the invention as defined in claim 1 applies mutatis mutandis to preferred embodiments of this coloring method preferred according to the invention with the stated incubation and exposure times and temperatures of the coloring composition.

Another preferred subject matter of the present invention is a method for the non-oxidative coloring of keratinous fibers, in particular human hair, in which a composition containing at least one anthocyanin-containing plant part and a second composition containing at least one glycosylase (E.C. 3.2) are mixed with one another, and this mixture is applied to the keratin fibers within a time of zero seconds to 10 minutes, preferably 10 seconds to 5 minutes, particularly preferably 15 to 60 seconds, and is rinsed off again after an exposure time of 1 to 60 minutes, more preferably 5 to 45 minutes, particularly preferably 20 to 35 minutes, extremely preferably 25 to 30 minutes, wherein the ready-to-use coloring composition has a temperature of 15-50° C., preferably 20-40° C., preferably 25-35° C., and wherein the keratin fibers are heated during the entire exposure time or at least during part of the exposure time. Heat is preferably supplied by a heat lamp, a drying hood, or a hair dryer. The heat supplied is within physiologically acceptable limits.

Preferably, the keratin fibers are dried after the composition according to the invention or preferred according to the invention has been rinsed out. Drying can take place without the active supply of heat. However, drying can also be carried out with a supply of heat at a temperature of 25 −120° C., particularly preferably at a temperature of 30-80° C., extremely preferably at a temperature of 35-60° C. Heat is preferably supplied by a heat lamp, a drying rod, a drying hood, a straightening iron, or a hair dryer.

The keratin fibers can be dried after each rinsing step using an absorbent cloth, such as a towel. The towel-dried hair can optionally also be dried partially or completely with a hairdryer or other heat source. It is also possible to let the keratin fibers air dry.

pH

Further preferred embodiments of the invention (coloring composition, coloring method) are characterized in that the composition according to the invention has a pH in the range of 1.0 to less than 4.0, preferably 1.5 to 3.5, particularly preferably 2.0 to 3.0, in each case measured at 20° C. In this acidic range of the coloring solution, a particularly good absorption of the dyes onto the keratin fibers was observed.

Further preferred embodiments of the invention (coloring composition, coloring method) are characterized in that the composition according to the invention comprises a cosmetic carrier.

In a first embodiment preferred according to the invention, the carrier is water. Further preferred embodiments of the invention (coloring composition, coloring method) are characterized in that the composition according to the invention contains, based upon its weight, water in an amount of 30.0-99.8 wt. %, preferably 50.0-98.0 wt. %, particularly preferably 70.0-90.0 wt. %, extremely preferably 80.0-86.0 wt. %.

In order to optimize the applicability of the composition and its dwell time on the keratin fibers, it is preferred for the composition according to the invention or preferred according to the invention to have a thickened consistency. Preferred compositions are in the form of gel, cream, or paste. Such carriers ensure a homogeneous distribution and a sufficient dwell time of the composition on the keratin fibers.

The compositions according to the invention and preferred according to the invention can optionally contain further additives in order to optimize the application properties of this composition. Preferred additives are in particular thickeners, which ensure that the composition stays better on the hair during application.

Compositions particularly preferably used according to the invention contain at least one or more hydrophilic thickeners, which are preferably selected from polysaccharides that are different from cellulose and can be chemically and/or physically modified. Hydrophilic thickeners that are particularly preferred according to the invention are compounds from the group of polysaccharides that are different from cellulose, since the skeletal structures of the polysaccharides are of natural origin and biodegradable. Preferred hydrophilic polysaccharide thickeners are different from celluloses, cellulose ethers, and cellulose esters and are selected from xanthan gum, alginic acids (and their corresponding physiologically acceptable salts, the alginates), agar agar (with the polysaccharide agarose present as the main component in agar agar), starch fractions and starch derivatives such as amylose, amylopectin, and dextrins, karaya gum, locust bean gum, gum arabic, pectins, dextrans, and guar gum, and mixtures thereof.

In preferred embodiments, the hydrophilic thickener present is xanthan gum, with a view to reliable viscosity adjustment and residue-free application to keratin fibers and the scalp.

Further preferred compositions according to the invention contain, with a view to reliable viscosity adjustment, at least one polymeric thickener selected from
non-ionic polymers such as vinylpyrrolidinone/vinyl acrylate copolymers, polyvinylpyrrolidinone, vinylpyrrolidinone/vinyl acetate copolymers, and polyethylene glycols with a molecular weight of 100,000 to 10,000,000 daltons;
cationic polymers, such as dimethyldiallyl ammonium chloride polymers, acrylamide-dimethyldiallyl ammonium chloride copolymers, dimethylaminoethylmethacrylate vinylpyrrolidinone copolymers quaternized with diethyl sulfate, vinylpyrrolidinone-imidazolinium-methochloride copolymers, and quaternized polyvinyl alcohol;
zwitterionic and amphoteric polymers, such as acrylamidopropyl-trimethylammonium chloride/acrylate copolymers and octylacrylamide/methyl methacrylate/tert-butylaminoethyl methacrylate/2-hydroxypropyl methacrylate copolymers, diallyldimethylammonium chloride/acrylate copolymers, t-butylaminoethyl methacrylate/N-(1,1,3,3-tetramethylbutyl) acrylamide/acrylate (/methacrylate) copolymers;
anionic homo- and copolymers based upon polyacrylic acids, polymethacrylic acids, cross-linked polyacrylic acids, cross-linked polymethacrylic acids, the esters and amides of possibly cross-linked poly (meth)acrylic acids, homo- and copolymers of acrylamido-2-methylpropanesulfonic acids, vinyl acetate/crotonic acid copolymers, vinylpyrrolidinone/vinyl acrylate copolymers, vinyl acetate/butyl maleate/isobornyl acrylate copolymers, methyl vinyl ether/maleic anhydride copolymers, and acrylic acid/ethyl acrylate/N-t-butyl acrylamide terpolymers, each in their acid or salt form.

Compositions that are particularly preferred according to the invention contain, in each case based upon their weight, at least one hydrophilic thickener in a total amount of 0.1 to 5 wt. %, preferably of 0.5 to 4 wt. %, more preferably of 1 to 3.5 wt. %, and very particularly preferably of 1.2 to 2 wt. %.

Compositions particularly preferred according to the invention contain at least one organic solvent that has a phenyl group in the molecule. Preferably, this solvent is selected from phenoxyethanol, benzyl alcohol, and mixtures thereof. Surprisingly, it was found that such aromatic solvents can have a positive effect on the coloring results of the coloring method according to the invention.

In a further preferred embodiment of the present invention, the compositions preferred according to the invention contain, in each case based upon their weight, 0.1 to 3 wt. %, preferably 0.5 to 2.5 wt. %, more preferably 0.8 to 1.0 wt. %, of at least one organic solvent that has a phenyl group in the molecule. In a further preferred embodiment of the present invention, the compositions according to the invention contain, in each case based upon their weight, 0.1 to 3 wt. %, preferably 0.5 to 2.5 wt. %, more preferably 0.8 to 1.0 wt. %, of at least one organic solvent selected from phenoxyethanol, benzyl alcohol, and mixtures thereof.

Further compositions that are particularly preferred according to the invention contain at least one aliphatic solvent selected from C₁-C₄ alkanols and C₂-C₁₂ polyols, in particular selected from ethanol, isopropanol, n-propanol, ethylene glycol, 1,2-propanediol, glycerin, diethylene glycol, dipropylene glycol, 1,3-butylene glycol, 1,2-hexanediol, 1,6-hexanediol, and 1,2-octanediol, and mixtures of these solvents.

Further compositions that are particularly preferred according to the invention contain, based upon their weight, at least one aliphatic solvent selected from C₁-C₄ alkanols and C₂-C₁₂ polyols, in a total amount of 0.01-60 wt. %, preferably 0.1-30 wt. %, particularly preferably 0.5-20 wt. %, extremely preferably 1-10 wt. %, further preferably 2-5 wt. %.

Other compositions that are particularly preferred according to the invention are characterized in that they do not contain an aliphatic solvent selected from C₁-C₄ alkanols and C₂-C₁₂ polyols.

Compositions particularly preferred according to the invention contain at least one oil. Preferred cosmetic oils are selected from natural and synthetic hydrocarbons, particularly preferably from paraffin oils, C₁₈-C₃₀ isoparaffins, in particular isoeicosane, polyisobutenes and polydecenes, C₈-C₁₆ isoparaffins, and 1,3-di-(2-ethylhexyl)cyclohexane; the benzoic acid esters of linear or branched C₈₋₂₂ alkanols; fatty alcohols having 6-30 carbon atoms that are unsaturated or branched and saturated or branched and unsaturated; triglycerides of linear or branched, saturated or unsaturated, optionally hydroxylated C₈₋₃₀ fatty acids, in particular natural oils; the dicarboxylic acid esters of linear or branched C₂-C₁₀ alkanols; the esters of the linear or branched, saturated or unsaturated fatty alcohols having 2-30 carbon atoms with linear or branched, saturated or unsaturated fatty acids having 2-30 carbon atoms, which can be hydroxylated; the addition products of 1 to 5 propylene oxide units to mono- or polyvalent C₈₋₂₂ alkanols; the addition products of at least 6 ethylene oxide units and/or propylene oxide units to mono- or polyvalent C₃₋₂₂ alkanols; the C₈-C₂₂ fatty alcohol esters of monovalent or polyvalent C₂-C₇ hydroxycarboxylic acids; the symmetrical, unsymmetrical, or cyclic esters of carbonic acid with C₃₋₂₂ alkanols, C₃₋₂₂ alkanediols or C₃₋₂₂ alkanetriols; the esters of dimers of unsaturated C₁₂-C₂₂ fatty acids (dimer fatty acids) with monovalent linear, branched, or cyclic C₂-C₁₈ alkanols or with polyvalent linear or branched C₂-C₆ alkanols; silicone oils, and mixtures of the aforementioned substances.

Other compositions that are particularly preferred according to the invention contain at least one surfactant or emulsifier.

Surfactants and emulsifiers in the sense of the present application are amphiphilic (bifunctional) compounds that consist of at least one hydrophobic and at least one hydrophilic molecule part. The hydrophobic group is preferably a hydrocarbon chain having 8-28 carbon atoms, which can be saturated or unsaturated, linear or branched. This C₈-C₂₈ alkyl chain is particularly preferably linear. The basic properties of the surfactants and emulsifiers are oriented absorption at boundary surfaces and also the aggregation to micelles and the formation of lyotropic phases. When selecting surfactants suitable according to the invention, it may be preferable to use a mixture of surfactants in order to optimally adjust the stability of the compositions according to the invention. Preferred surfactants and emulsifiers are selected from anionic, cationic, zwitterionic, amphoteric, and nonionic surfactants and emulsifiers and mixtures of these substances.

Other compositions that are particularly preferred according to the invention contain at least one linear saturated alkanol having 12-30 carbon atoms.

Preferred linear saturated alkanols having 12-30 carbon atoms, in particular having 16-22 carbon atoms, are selected from cetyl alcohol, stearyl alcohol, arachidyl alcohol, behenyl alcohol, and lanolin alcohol, and mixtures of these alkanols. Alkanol mixtures that are particularly preferred according to the invention are those obtained from the industrial hydrogenation of vegetable and animal fatty acids. Preferably, the total amount of at least one linear saturated alkanol having 12-30 carbon atoms is 0.1-20 wt. %, preferably 0.5-16.5 wt. %, and particularly preferably 3-10 wt. %, in each case based upon the weight of the composition according to the invention.

Preferred compositions and coloring methods according to the invention are further characterized in that no hydrogen peroxide is used in them.

In order to make the compositions according to the invention also olfactorily attractive for the user, further compositions particularly preferred according to the invention are characterized in that they contain at least one fragrance compound or odoriferous compound.

Compositions that are extremely preferred according to the invention are characterized in that they contain, based upon their weight, at least one fragrance in a total amount of 0.01-5 wt. %, preferably 0.1-3 wt. %, particularly preferably 0.5-2 wt. %, extremely preferably 1-1.5 wt. %.

What has been said regarding preferred embodiments of the coloring method according to the invention applies mutatis mutandis to preferred embodiments of the coloring compositions according to the invention.

### EXEMPLARY EMBODIMENTS

The exemplary embodiments presented below are intended to explain the subject matter of the invention in more detail, without limiting it thereto.

The coloring method according to the invention was carried out on strands of white buffalo belly hair (tied in a round shape, approx. 8 cm of free hair).

As a test coloring agent, a 1 wt. % solution of a powder of dried Ribes nigrum berries in water, adjusted to a pH of 3±0.15 with 10 wt. % hydrochloric acid, measured at 20° C., was prepared and used for all the coloring experiments described below.

The raw material used was “Black Currant Fruit Powder” from Matha Exports International LLP, Ashram, New Delhi, India. According to the manufacturer, these are powdered, spray-dried blackcurrants (Ribes nigrum berries). The manufacturer states that this raw material contains 5.25 wt. % of anthocyanins, based upon the Ribes nigrum berry powder.

### Standard Coloring Method for all the Coloring Tests Described Below

Buffalo belly hair strands were immersed in the respective coloring solution for 30 minutes while stirring. The liquor ratio (amount of Ribes nigrum solution per gram of hair) was 50 mL Ribes nigrum solution per gram of hair.

Unless otherwise stated, the coloring solution had a temperature of 20° C. (room temperature).

After coloring, the hair strands were rinsed under running deionized water for 30 seconds and combed 20 times (20° C.).

The hair strands were then dried with a commercially available hair dryer at a defined distance (d=10 cm) and a defined temperature (T=80±5° C.) while combing them 20 times. The strands were then colorimetrically measured.

### Determination of the Hue Achieved by the Method According to the Invention

All colorimetric measurements were carried out using the Spectraflash SF 600 colorimetric device from Datacolor.

The color difference, also known as dE or ΔE, can be easily determined colorimetrically using a colorimeter that measures colors in the L*, a*, b* color space—for example, a Datacolor colorimeter, type Spectraflash SF 600.

The L*, a*, b* color space refers to the CIELAB color space. The L value represents the brightness of the color (black-white axis); the greater the L value, the brighter the color. The a value represents the red-green axis of the system; the greater this value, the more the color is shifted towards red. The b value represents the yellow-blue axis of the system; the greater this value, the more the color is shifted towards yellow.

The color shift ΔE, i.e., the color difference between two (hair) colors, for each of which an L*, a*, b* value combination was determined, is calculated according to the following formula:

$$Δ E = {( Δ {L}^{2} + Δ {a}^{2} + Δ {b}^{2} )}^{0.5}$$

The greater the value for ΔE, the more pronounced the color difference.

For the spectrophotometer measurements, a D65 lamp and a diffuse/8° optical configuration were used. The spectral reflectance data for each sample from 380 nm to 700 nm were converted to colorimetric data using DCI Color software. Reflectance measurements were determined for each hair sample, wherein the average of 4 measurements was recorded.

The color difference (ΔE) between uncolored strand and colored strand was calculated according to the following formula:

$$Δ E = \sqrt{{( Lv - Ln )}^{2} + {( av - an )}^{2} + {( bv - bn )}^{2}} , where$$

$$Lv , av , bv : colorimetric values for colored strand$$

$$Ln , an , bn : colorimetric values for uncolored , untreated strand$$

Coloring with Ribes nigrum without Enzyme Addition

In a first investigation, the hair was colored—not according to the invention—only with Ribes nigrum, without enzyme addition. The influence of the temperature of the coloring solution on the resulting coloration was also investigated. The colorimetric values are listed in table 1.

TABLE 1
Color results of the colorations in 1 wt. % Ribes nigrum
solution without enzyme addition at various temperatures
of the coloring solution; 30 minutes exposure time, pH 3

| | | a* | b* | |
|---|---|---|---|---|
| | L* | green-red axis | blue-yellow axis | ΔE |
| Buffalo hair, | 80.08 | −0.97 | 8.48 | |
| untreated | | | | |
| Room | 66.97 | 14.58 | 9.18 | 21.94 |
| temperature | | | | |
| (20° C.) | | | | |
| 30° C. | 65.46 | 15.27 | 12.79 | 22.27 |
| 40° C. | 61.95 | 16.86 | 20.48 | 28.12 |
| 45° C. | 56.68 | 20.81 | 23.91 | 35.50 |
| 50° C. | 54.93 | 21.59 | 27.11 | 18.94 |
| 60° C. | 57.73 | 20.24 | 40.96 | 44.77 |
| 70° C. | 62.74 | 15.01 | 45.68 | 32.99 |

The ΔE values refer to the L*, a*, b* values of the untreated buffalo hair.

The achieved colorings ranged from gray-brown to light brown to dark blond. It was found that the intensity of the coloring, represented by the L* value, increased with increasing temperature of the coloring solution and reached a maximum at 50° C., i.e., the lowest L* value was observed at 50° C. The greatest temperature dependence was observed along the blue-yellow axis, represented by the colorimetric b* value. As the temperature of the coloring solution increased, the b* value increased, meaning that the coloring shifted towards yellow.

### Color Change by Addition of Glycosylases

Surprisingly, it was found that the affinity of dyes from anthocyanin-containing plant parts to the keratin fiber and the resulting coloration depends largely upon the sugar residues and the molecular size and charge of the anthocyanins. As a result of the addition of glycosylases, sugar residues can be split off, and the aglycones, the so-called anthocyanidins, which are actually responsible for the color, can be formed. In this way, more and different anthocyanins present in Ribes nigrum, in particular in the berries of Ribes nigrum, can bind to the keratin fiber compared to coloring without the addition of glycosylase.

In the experiment, a glycosylase mixture (E.C. 3.2) was used, which was obtained from Novozymes. This enzyme mixture comprised at least one endo-1,4-beta-glucanase (E.C. 3.2.1.4) in a total amount of 20-35 wt. %, at least one beta-glucosidase (E.C. 3.2.1.21) in a total amount of 15-25 wt. %, furthermore at least one enzyme selected from at least one exo-1,4-beta-D-glucanase (E.C. 3.2.1.91) and at least one oligoxyloglucan reducing-end-specific cellobiohydrolase (E.C. 3.2.1.150) and mixtures thereof in a total amount of 25-35 wt. %, as well as at least one other enzyme with glycosylase activity in a total amount up to 100 wt. %, wherein the indicated amounts are relative to the total weight of the enzyme mixture.

The enzyme active substance content in the mixture used, which was present as an aqueous composition, was 190 μg/μL.

For the colorings with Ribes nigrum in combination with the previously described glycosylase mixture, a 1 wt. % solution of a powder of dried Ribes nigrum berries in water, to which the respective amount of glycosylase mixture was added and which was adjusted to a pH of 3±0.15, measured at 20° C., with 10 wt. % hydrochloric acid, was prepared and used.

The strands were colored according to the standard coloring method described above. For the series of experiments shown in table 2, the strands were colored in the acidic Ribes nigrum solution directly after the enzyme was added thereto.

Like the colorimetric values presented in table 2 show, the addition of different amounts of glycosylase mixture led to an intensification of the violet hue in the solution and on the hair fiber. The most intense color result was achieved with 10 μL enzyme solution/50 mL coloring solution, i.e., with a concentration of enzyme active substance of 1.9 mg enzyme active substance/50 mL.

TABLE 2
Color results of the colorations in 1 wt. % Ribes nigrum solution
with various concentrations of glycosylase solution (cellulase
solution), 30 minutes exposure time, pH 3, room temperature

| | L* | a* | b* | ΔE |
|---|---|---|---|---|
| Buffalo hair, | 80.08 | −0.97 | 8.48 | |
| untreated | | | | |
| 1 μL cellulase | 57.92 | 17.54 | −7.9 | 33.2 |
| 10 μL cellulase | 53.17 | 20.39 | −10.38 | 39.19 |
| 50 μL cellulase | 58.43 | 17.64 | −8.43 | 33.18 |
| 100 μL cellulase | 58.14 | 16.58 | −6.13 | 31.66 |
| 200 μL cellulase | 58.30 | 17.60 | −7.89 | 32.97 |

The ΔE values refer to the L*, a*, b* values of the untreated buffalo hair.

In a further series of experiments, it was investigated whether it is necessary to allow the glycosylase to act upon the anthocyanin-containing plant parts for a certain incubation time first before coloring the keratin fibers. The colorimetric measurement values shown in table 3 show that no incubation time is necessary, but that the shortest possible incubation time is best for the color result (cf. Table 3).

In this context, incubation time is understood to be the time between the addition of the enzyme solution to the Ribes nigrum solution and the addition of the keratin fibers to be colored.

The strands were treated according to the standard coloring method described above. For the series of experiments shown in table 3, the strands were either colored in the acidic Ribes nigrum solution directly after the addition of the enzyme thereto (incubation time 0 minutes) or added to the coloring solution only after the specified incubation time had elapsed and then treated according to the standard coloring method.

TABLE 3
Color results of the colorations in 1 wt. % Ribes nigrum
solution in combination with 10 μL glycosylase solution
(cellulase solution) after different incubation times,
30 minutes exposure time, pH 3, room temperature

| | L* | a* | b* | ΔE |
|---|---|---|---|---|
| Buffalo hair, | 80.08 | −0.97 | 8.48 | |
| untreated | | | | |
| 0 min | 53.17 | 20.39 | −10.38 | 39.19 |
| 15 min | 54.99 | 17.57 | −7.74 | 35.16 |
| 30 min | 57.03 | 16.70 | −7.16 | 32.99 |
| 45 min | 55.79 | 16.59 | −6.84 | 33.66 |
| 60 min | 56.76 | 15.67 | −5.50 | 31.88 |

The ΔE values refer to the L*, a*, b* values of the untreated buffalo hair.

In a further series of experiments, the influence of the temperature of the coloring solution on the resulting coloration in the presence of the added glycosylases was investigated. The colorimetric values are listed in table 4.

TABLE 4
Color results of the colorations of 1 wt. % Ribes nigrum solution
with 100 μL glycosylase solution (cellulase solution) at various
temperatures of the coloring solution, 30 minutes exposure time, pH 3

| | L* | a* | b* | ΔE |
|---|---|---|---|---|
| Buffalo hair, | 80.08 | −0.97 | 8.48 | |
| untreated | | | | |
| Room | 58.14 | 16.58 | −6.13 | 31.66 |
| temperature | | | | |
| (20° C.) | | | | |
| 30° C. | 53.0 | 18.72 | −6.73 | 36.77 |
| 40° C. | 48.22 | 18.87 | −3.09 | 39.28 |
| 50° C. | 41.36 | 21.27 | 0.02 | 45.45 |
| 60° C. | 38.80 | 20.64 | 11.36 | 46.7 |
| 70° C. | 41.89 | 19.78 | 27.54 | 47.46 |

The ΔE values refer to the L*, a*, b* values of the untreated buffalo hair.

The resulting colorings ranged over various violet hues, which initially became darker and then turned into brown hues.

Here, too, the greatest temperature dependence was observed along the blue-yellow axis, represented by the colorimetric b* value. As the temperature of the coloring solution increased, the b* value in particular increased, meaning that the coloring shifted towards yellow. The L* value also showed a clear temperature dependence, however.

1. A method for non-oxidative coloring of keratinous fibers, the method comprising:
applying a composition comprising at least one anthocyanin-containing plant part and at least one glycosylase outside the at least one anthocyanin-containing plant part to the keratinous fibers; and
rinsing the composition off the keratinous fibers after an exposure time.

2. The method of claim 1, wherein the at least one glycosylase is at least one glycosidase, (E.C. 3.2:1).

3. The method of claim 2, wherein the at least one glycosidase is at least one cellulase.

4. The method of claim 1, wherein the at least one glycosylase is selected from the group consisting of endo-1,4-beta-glucanase, beta-glucosidase, exo-1,4-beta-glucosidase, cellulose-1,4-beta-cellobiosidase, exo-1,4-beta-D-glucanase, oligoxyloglucan reducing-end-specific cellobiohydrolase, and mixtures thereof.

5. The method of claim 1, wherein:
the composition comprises an enzyme mixture comprising the at least one glycosylase, and
the enzyme mixture comprises:
at least one endo-1,4-beta-glucanase in an amount of from 20-35 wt. % based on the total weight of the enzyme mixture,
at least one beta-glucosidase in a total in an amount of from 15-25 wt. % based on the total weight of the enzyme mixture,
at least one exo-1,4-beta-D-glucanase (E.C. 3.2.1.91) in a total in an amount of from 25-35 wt. % based on the total weight of the enzyme mixture, and
at least one other enzyme with cellulase activity in an amount up to 100 wt. % based on the total weight of the enzyme mixture.

6. The method of claim 1, wherein the at least one glycosylase is in an amount of from 0.0001-1 wt. % based on the total weight of the composition.

7. The method of claim 1, wherein the at least one anthocyanin-containing plant part is selected from the group consisting of Ribes nigrum, Euterpe oleracea (cabbage palm, açai), Aronia (chokeberry), Malus domestica, Solanum melongena, Fragaria, Vaccinium, Hibiscus, Sambucus nigra, Citrus sinensis, Rubus sect. Rubus, Rubus idaeus, Prunus avium, Prunus cerasus, Ribes rubrum, red cabbage (Brassica oleracea convar. capitata var. rubra L.), and Vitis vinifera.

8. The method of claim 1, wherein the at least one anthocyanin-containing plant part is selected from the group consisting of berries, fruits, aggregate nut fruits, aggregate drupes, drupes, pomes, peels, cones, leaves, stems, branches, bark, trunk wood, seeds, and roots of an anthocyanin-containing plant.

9. The method of claim 1, wherein the at least one anthocyanin-containing plant part is used as in the form of a water-soluble powder.

10. The method of claim 1, wherein the at least one anthocyanin-containing plant part is in the form of the anthocyanin-containing plant part.

11. The method of claim 1, wherein the composition comprises the at least one anthocyanin-containing plant part in an amount of from 0.05-20 wt. % based on the total weight of the composition.

12. The method of claim 1, wherein the composition has a pH of from 1.0 to less than 4.0, measured at 20° C.

13. The method of claim 1, further comprising:
mixing the at least one anthocyanin-containing plant part and the at least one glycosylase to form the composition,
wherein the composition is applied to the keratinous fibers within a time of zero seconds to 10 minutes after forming the composition, and
wherein the exposure time is from 1 to 60 minutes.

14. A composition for non-oxidative coloring of keratinous fibers, the composition comprising:
at least one anthocyanin-containing plant part; and
at least one glycosylase outside the at least one anthocyanin-containing plant part.

15. (canceled)

16. The method of claim 1, wherein the keratinous fibers are human hair.

17. The composition of claim 14, wherein the keratinous fibers are human hair.

18. The method of claim 6, wherein the at least one glycosylase is in an amount of from 0.01-0.03 wt. % based on the total weight of the composition.

19. The method of claim 11, wherein the composition comprises the at least one anthocyanin-containing plant part in an amount of from 1-1.5 wt. % based on the total weight of the composition.

20. The method of claim 13, wherein the composition is applied to the keratinous fibers within a time of 15 to 60 seconds after forming the composition.

21. The method of claim 13, wherein the exposure time is from 25 to 30 minutes.
